(19) United States
(12) Patent Application Publication
(10) Pub. No.: US 2025/0135089 A1
(43) Pub. Date: May 1, 2025

(54) SENSOR ASSEMBLY FOR PRESSURE SENSING AND BACK-UP FLUID SUPPLY IN SURGICAL CONSOLES

(71) Applicant: Alcon Inc., Fribourg (CH)

(72) Inventor: Vincent A. BAXTER, Temecula, CA (US)

(21) Appl. No.: 18/817,727

(22) Filed: Aug. 28, 2024

Related U.S. Application Data
(60) Provisional application No. 63/594,779, filed on Oct. 31, 2023.

(51) Int. Cl. A61M 1/00

(52) U.S. Cl. CPC A61M 1/74; A61M 2205/3348

(57) ABSTRACT

A back-up infusion pressure sensor module for use in an ophthalmic surgical console includes a squeeze plate configured to engage with a fluid bag. The back-up infusion pressure sensor module further includes a pressure sensor assembly, having a sensor door and a sensor frame, wherein the sensor door pivots relative to a pin coupled to a top portion of the pressure sensor assembly, configured to cause the sensor door to be biased towards the fluid bag. The pressure sensor assembly comprises a base mounted to the back side of the sensor frame of the pressure sensor assembly, one or more springs, a spring bracket coupled to the one or more springs, and a force sensor in contact with the spring bracket, configured to determine a pressure force against the fluid bag.

### CROSS-REFERENCE TO RELATED APPLICATIONS

This application claim benefit of and priority to U.S. Provisional Patent Application No. 63/594,779, filed Oct. 31, 2023, which is hereby assigned to the assignee hereof and hereby expressly incorporated by reference in its entirety as if fully set forth below and for all applicable purposes.

### INTRODUCTION

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the iris and/or lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed.

During anterior and/or posterior segment surgery, tissue fragments and other materials may be aspirated or suctioned out of the eye through, e.g., a hollow needle or cannula. Also, during the procedure, a fluid may be irrigated or infused into the eye to maintain an intraocular pressure (IOP) and eye chamber stability. A fluidics module of a surgical console is typically used to facilitate the aspiration/suction and irrigation/infusion functionalities described above.

Balancing fluid irrigation/infusion and aspiration/suction to maintain intraocular pressure is important to the success of an ophthalmic surgical procedure. Generally, intraocular pressure needs to be maintained at a relatively constant pressure in order to avoid adverse effects to the patient's eye. It is therefore desirable to accurately and efficiently monitor the flow or pressure of fluids being introduced into the eye, as well as the pressure of the fluids being aspirated/suctioned from the eye, during ophthalmic surgical procedures.

### SUMMARY

Aspects of the present disclosure relate to driving and monitoring fluid infusion or irrigation for ophthalmic (eye) procedures, and more specifically, squeeze plates and external pressure sensors for infusion or irrigation fluid bags used in ophthalmic surgical procedures.

In certain embodiments, a back-up infusion pressure sensor module is provided. The back-up infusion pressure sensor module includes a squeeze plate configured to engage with a fluid bag. The back-up infusion pressure sensor module further includes a pressure sensor assembly having a sensor door and a sensor frame, wherein the sensor door pivots relative to a pin coupled to a top portion of the pressure sensor assembly, configured to cause the sensor door to be biased towards the fluid bag. The pressure sensor assembly comprises a base mounted to the back side of the sensor frame of the pressure sensor assembly, one or more springs, a spring bracket coupled to the one or more springs, and a force sensor in contact with the spring bracket, configured to determine a pressure force against the fluid bag.

In certain embodiments, a back-up infusion pressure sensor module is provided. The back-up infusion pressure sensor module includes a squeeze plate configured to engage with a fluid bag. The back-up infusion pressure sensor module further includes a pressure sensor assembly having a first portion and a second portion, wherein the first portion pivots relative to a pin coupled to the second portion and the pressure sensor assembly is configured to cause at least one portion of the pressure sensor assembly to contact the fluid bag. The pressure sensor assembly further includes a base mounted to the back side of the pressure sensor assembly, one or more biasing members, a bracket coupled to the one or more biasing members, and a force sensor in contact with the bracket, configured to determine a pressure source against the fluid bag.

### BRIEF DESCRIPTION OF THE DRAWINGS

The drawings described herein are for illustrative purposes only, are schematic in nature, and are intended to be exemplary rather than to limit the scope of the disclosure.

FIG. 1A illustrates an example ophthalmic surgical system that may be used to perform ophthalmic procedures on an eye, according to embodiments described herein.

FIG. 1B illustrates example subsystems of a surgical console of the ophthalmic surgical system of FIG. 1A, according to embodiments described herein.

FIGS. 2A-2B illustrate perspective views of a portion of a fluidics subsystem of a surgical console and a fluid bag module thereof, according to embodiments described herein.

FIGS. 3A-3D illustrate perspective views of exemplary pressure sensor assembly, according to embodiments described herein.

FIG. 4A illustrates a detailed cross-sectional view of an exemplary pressure sensor assembly in an uncompressed position, according to embodiments described herein.

FIG. 4B illustrates a detailed cross-sectional view of an exemplary pressure sensor assembly in a compressed position, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to backup fluid infusion or irrigation for ophthalmic (eye) procedures, and more specifically, fluid bag pressure sensors that incorporate a means for backup infusion or irrigation for use with fluid bags used in ophthalmic surgical procedures.

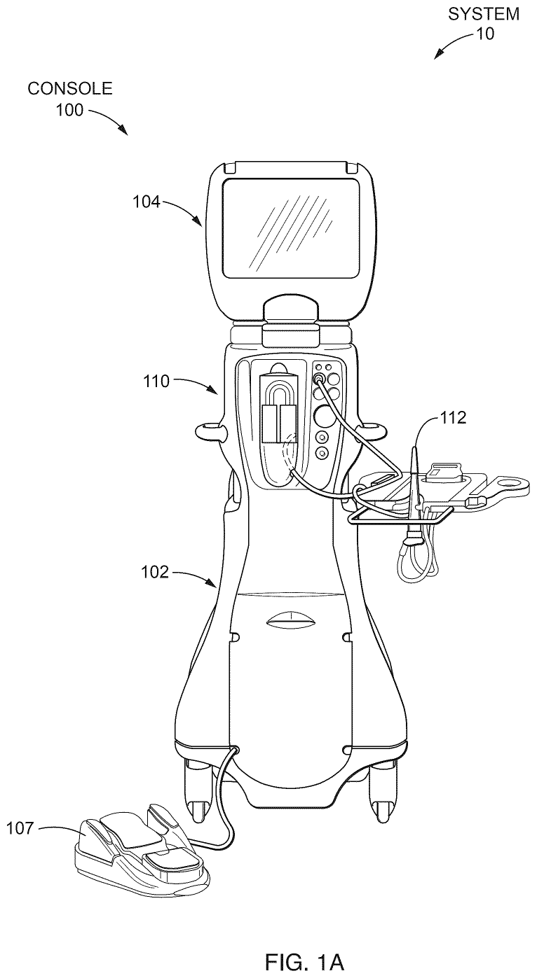

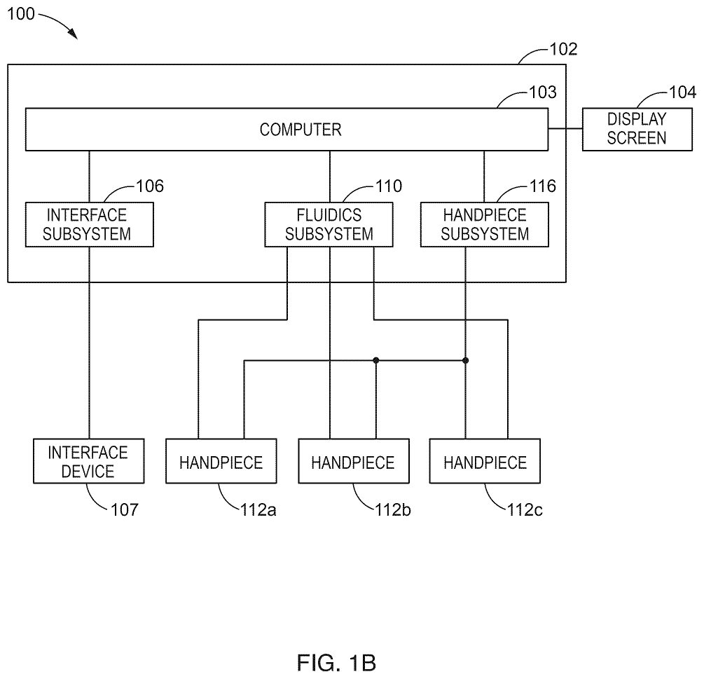

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), an interface device 107 (e.g., a foot pedal), and a hand piece 112. Console 100 includes a housing 102, a display screen 104, and a fluidics subsystem 110. The components of system 10 and console 100 may be coupled as shown and described in more detail with reference to FIG. 1B.

FIG. 1B illustrates example subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and hand pieces 112 (112a-c). An interface device 107 receives input to console 100, sends output from console 100, and/or processes the input and/or output. Examples of an interface device 107 include a foot pedal, manual input device (e.g., a keyboard), and a display. Interface subsystem 106 receives input from and/or sends output to interface device 107.

Fluidics subsystem 110 provides fluid control for one or more hand pieces 112 (112a-c). For example, fluidics subsystem 110 may manage fluid for an infusion or irrigating cannula. In some embodiments, fluidics subsystem 110 may be operatively coupled to a surgical cassette during a surgical procedure. For example, a surgical cassette may be inserted into, attached to, and/or integrated with fluidics subsystem 110 via a coupling mechanism. The coupling mechanism may comprise one or more of a latching mechanism, locking mechanism, or other similar connection mechanism. When fluidics subsystem 110 is operatively coupled to the surgical cassette, fluidics subsystem 110 may control irrigation/infusion and/or aspiration/suction of fluids through the surgical cassette. In certain embodiments, the fluidics subsystem 110 includes one or more mechanical pumps having roller pump heads configured to engage with one or more corresponding pump assemblies on the surgical cassette. The engagement of the roller pump heads and pump assemblies generates a source of pressure and/or vacuum utilized during an ophthalmic surgical procedure.

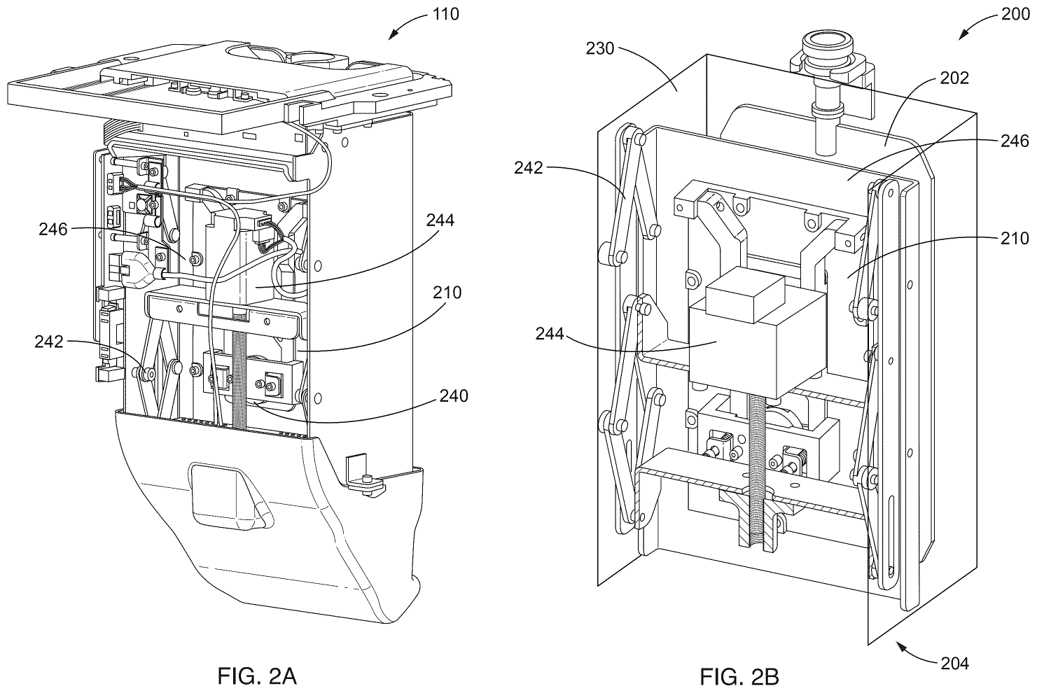

Further, fluidics subsystem 110 may include a fluid bag (e.g., fluid bag 202 shown in FIG. 2) that acts as a fluid source for providing fluid to the surgical cassette (e.g., provides fluid to the mechanical pump engaging with one or more pump assemblies on the surgical cassette). The fluidics subsystem 110 has one or more components engaged with the fluid bag 202 that control fluid flow supplied to the surgical cassette and/or hand piece 112 to provide infusion/irrigation to the eye during a surgical procedure. The fluidics subsystem 110 is configured to receive the fluid bag 202 in a cavity, slot, or other receptacle of the fluidics subsystem 110.

Hand piece 112 may be any suitable ophthalmic surgical instrument, e.g., an ultrasonically-driven phacoemulsification (phaco) hand piece, a laser hand piece, an irrigating cannula, a vitrectomy hand piece, or another suitable surgical hand piece. The hand piece 112 may be communicatively coupled to the surgical console 100 through fluid or electrical communication. One or more connections may exist between the hand piece 112 and the surgical console 100, such as tubing configured to carry aspiration and/or infusion fluid between the console and the hand piece.

Hand piece subsystem 116 supports one or more hand pieces 112. For example, hand piece subsystem 116 may manage ultrasonic oscillation for a phaco hand piece, provide laser energy to a laser hand piece, control operation of an irrigating cannula, and/or manage features of a vitrectomy hand piece.

Computer 103 controls operation of ophthalmic surgical system 10. Generally, computer 103 includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C, C++, Java, Python, Rust, or any other programming language (including assembly languages, hardware description languages, and database programming languages). In some instances, the code may be a program that, when loaded into the processor, causes the surgical console 100 to receive and process information from one or more of subsystems 106, 110, and 116, (for example, providing fluid control for one or more hand pieces 112 or other devices in communication with the surgical console 100).

The processor may be, or include, a microprocessor, a microcontroller, an embedded microcontroller, a programmable digital signal processor, or any other programmable device operable to receive information from the memory or other devices in communication with the processor, computer 103, and/or console 100, and perform one or more operations on the received information. For example, the processor may send instructions to components of fluidics subsystem 110, or other devices or systems in communication with computer 103, for controlling such devices and systems. The processor may also be operable to output results based on the operations performed thereby. In certain embodiments, computer 103 includes a controller that sends instructions to components of system 10. A display screen 104 shows data provided by computer 103.

FIGS. 2A-2B illustrate perspective views of a portion of fluidics subsystem 110 of surgical console 100 and a fluid bag module 200 thereof, respectively, according to embodiments described herein. The fluidics subsystem 110 includes the fluid bag module 200, which further has a fluid bag pressure module 204 that can be disposed within, or in operable communication with, other components of surgical console 100. A removable fluid bag 202 is shown as being disposed in the fluid bag module 200 in FIG. 2B.

The fluid bag pressure module 204, as shown in FIGS. 2A-2B, includes a squeeze plate 246, a back-up infusion/irrigation pressure sensor assembly 210 (referred to herein as “pressure sensor assembly”) attached to the squeeze plate 246, and a fluid bag chamber 230 adjacent to the squeeze plate 246 and pressure sensor assembly 210.

In order to control the rate at which infusion and/or irrigation fluid is provided to the fluidics subsystem 110, the console 100 automatically controls the amount of pressure the squeeze plate 246 provides to the fluid bag 202. During ophthalmic procedures, the squeeze plate 246 is actuated against the fluid bag 202 to squeeze the bag and force infusion and/or irrigation fluid out of the fluid bag 202 for delivering fluid to the eye of the patient. The back-up infusion/irrigation pressure sensor assembly 210 is configured to monitor the pressure of the squeeze plate 246 against the fluid bag 202 and/or provide back-up pressure on the fluid bag 202 in the event of loss of power to the squeeze plate 246.

The fluid bag chamber 230 includes a compartment defined by one or more walls formed of any suitable materials. For example, the walls of fluid bag chamber 230 may include thin sheets of metal alloy, such as stainless steel, for example, which can also be utilized to provide support to the fluid bag 202 on a side of the fluid bag 202 opposite from the squeeze plate 246, in addition to the squeeze plate 246. In certain embodiments, the fluid bag chamber 230 is at least partially defined by the squeeze plate 246.

In certain embodiments, the squeeze plate 246 may be configured to respond to movement of a drive assembly 244 such that the entire squeeze plate 246, and the pressure sensor assembly 210 coupled thereto, translate in a first direction toward the fluid bag 202, or a second direction away from the fluid bag 202, when the drive assembly 244 is activated. Together with a translation assembly 242, which can include one or more sets of cross-linked bars, the drive assembly 244 drives lateral movement of the squeeze plate 246, as well as the pressure sensor assembly 210, against or away from the fluid bag 202. As shown in FIGS. 2A-2B, the drive assembly 244 may include a motorized lead screw actuator (e.g., a leadscrew coupled to a stepper motor); however, other types of linear actuators are also contemplated. In certain embodiments, the drive assembly 244 may be driven using pneumatic pressure in response to commands automatically received from the surgical console 100, for example.

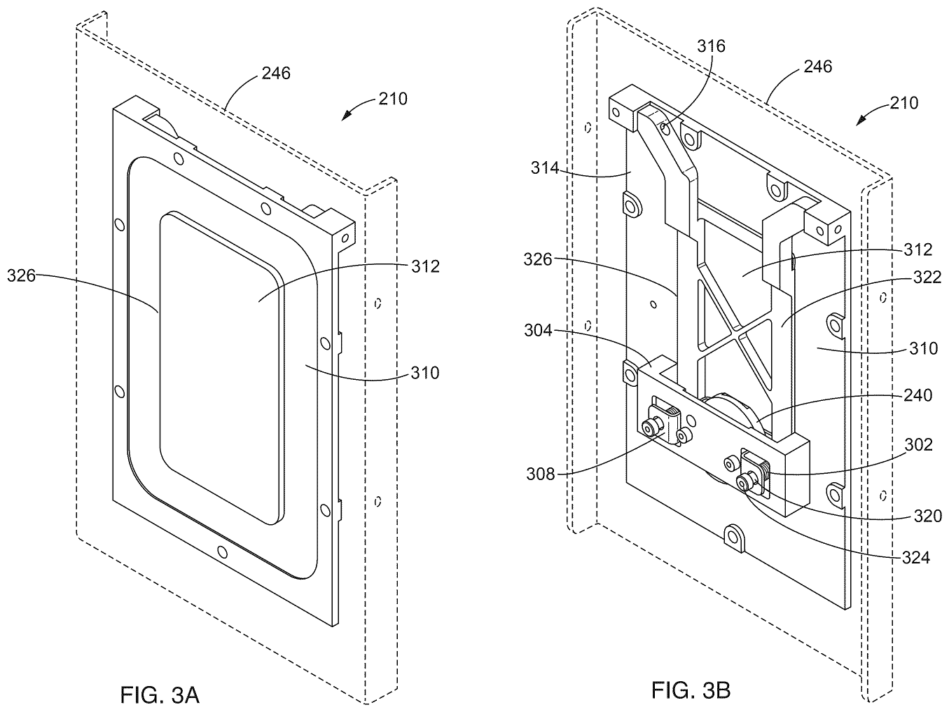

FIGS. 3A-3D illustrate perspective views of the pressure sensor assembly 210, according to embodiments described herein. In certain embodiments, the pressure sensor assembly 210 includes a sensor door 312, a sensor door support 322 coupled to and supporting a back side of the sensor door 312, a sensor frame 310 coupled to and disposed around the sensor door support 322, a force sensor 240 disposed through the sensor door 312, one or more springs 302 acting on the sensor door 312, one or more spring pins 320 disposed through the springs 302, a spring bracket 308 disposed around the force sensor 240, and a sensor support base 304 disposed on a back side of the sensor frame 310.

The pressure sensor assembly 210 may couple to the squeeze plate 246 via one or more brackets, screw holes, or bolts in the sensor frame 310. The sensor frame 310 may be configured to be centrally disposed through an opening in the center of the squeeze plate 246. The sensor frame 310 may be disposed with the front side of the sensor frame 310 coplanar with the front side of the squeeze plate 246, or the front side of the sensor frame 310 may be disposed in front or behind the front side of the squeeze plate 246. Thus, upon translation of the squeeze plate 246 towards the fluid bag 202, the sensor frame 310, and therefore the pressure sensor assembly 210, may also translate towards the fluid bag 202.

The fluid bag 202 may be removably placed in the surgical console 100, behind the squeeze plate 246, as demonstrated in FIG. 2B, prior to or during the performance of an ophthalmic surgical procedure. As described herein, the front side refers to a side or surface of a component closer to, or facing toward, a fluid bag in the module, while the back side refers to a side or surface of a component further from, or facing away from, a fluid bag in the module.

The fluid bag 202 may be coupled to the fluidics subsystem (e.g., through tubing) to provide fluid to a surgical cassette, surgical hand piece, and/or other device used in the surgical procedure. A front side of the pressure sensor assembly 210, and more particularly, a front side of the sensor door 312, is configured to contact the fluid bag 202 on one side of the fluid bag 202 to squeeze the fluid bag 202, while a wall of the fluid bag chamber 230 is configured to contact and provide a resisting force against the opposite side of the fluid bag 202. Accordingly, the front side of the sensor door 312 may be sized and shaped to provide an adequate “squeezing surface” for squeezing, or providing pressure against, the fluid bag 202 to force infusion/irrigation fluid therefrom. In certain embodiments, the front side of the sensor door includes a substantially planar surface, as shown in FIG. 3A. However, other configurations of the sensor door 312 are also contemplated.

A back side of the sensor door 312 is coupled the sensor door support 322. In certain embodiments, the sensor door 312 and sensor door support 322 are separate components; in other embodiments, the sensor door 312 and sensor door support 322 are integrated in a single component. The sensor door support 322 indirectly and movably couples the sensor door 312 to the sensor frame 310. For example, in the embodiments of FIGS. 3A and 3B, the sensor door support 322 is movably connected to one or more pins 316 at a top portion 314 of the back side of the sensor frame 310. The one or more pins 316 may each comprise an axis disposed parallel to a plane of the front side of the sensor door 312 and perpendicular to a direction of linear motion of the squeeze plate 246, which allows the sensor door support 322, and the sensor door 312 coupled thereto, to pivot about the axis of the pin(s) 316 and through an opening 326 in the sensor frame 310. The pin(s) 316 secure the sensor door support 322 and the sensor door 312 to the sensor frame 310. In use, as the squeeze plate 246 and the pressure sensor assembly 210 are moved linearly toward the fluid bag 202, the sensor frame 310 stays set in rotational orientation relative to the axis of the pin(s) 316. However, the pin(s) 316 facilitate rotation of the sensor door support 322 and the sensor door 312 about the axis of the pin(s) 316, thereby enabling the sensor door 312 to pivot relative to the sensor frame 310 and through the opening 326. Note that, although pins 316 are described, other types of rotational couplings or connectors for facilitating pivoting of the sensor door 312 through the opening 326 are also contemplated.

The sensor door 312, the sensor door support 322, and/or the sensor frame 310 may be formed of a metal or metal alloy materials, such as stainless steel or aluminum, for example. In certain embodiments, the sensor door and/or the sensor frame 310 are formed of thermoplastic polymer materials, or other plastic materials. In certain embodiments, the sensor door 312, the sensor door support 322, and/or the sensor frame 310 are formed of the same materials; in certain embodiments, the sensor door 312, the sensor door support 322, and/or the sensor frame 310 are formed of different materials.

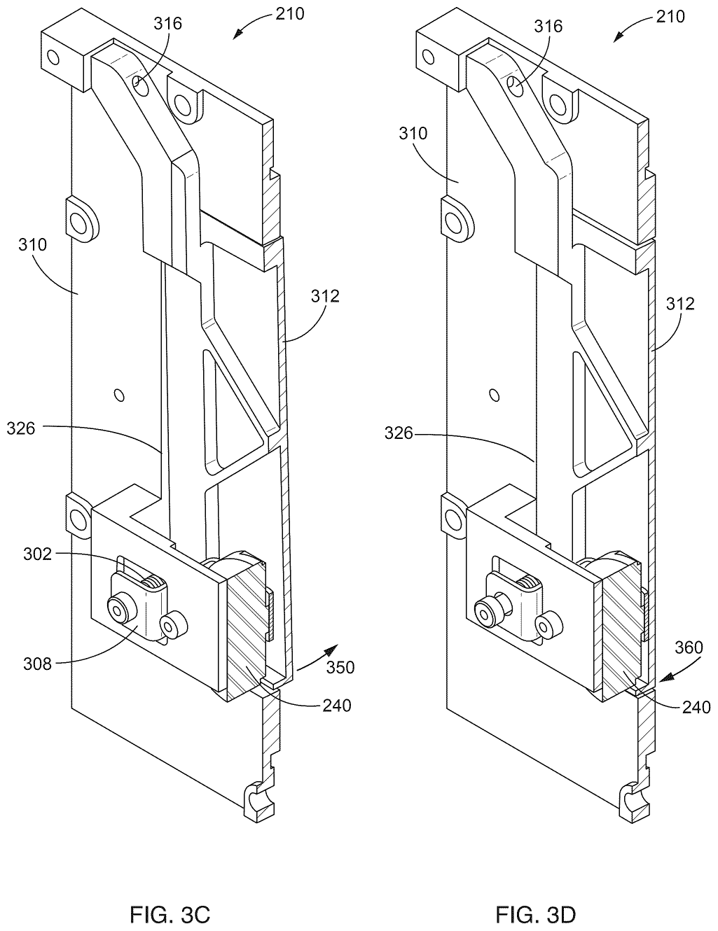

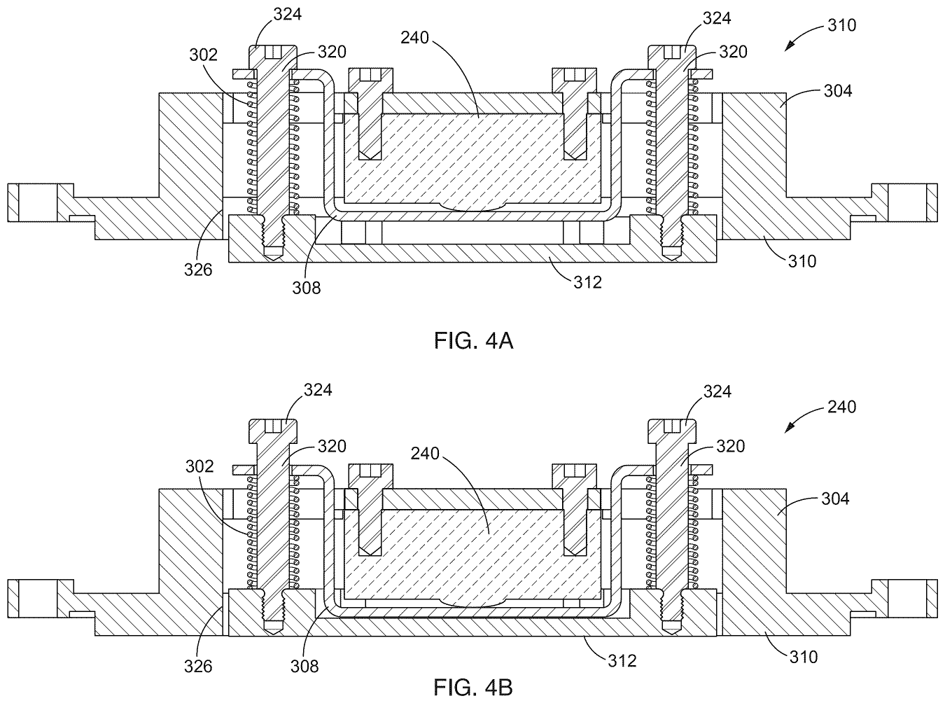

The sensor door 312 is further coupled to the force sensor 240, which is mounted to the sensor support base 304 disposed on the back side of the sensor frame 310 and thus, the back side of the pressure sensor assembly 210. Responsive/opposing forces from the fluid bag 202 acting against the sensor door 312 as the pressure sensor assembly 210 is translated towards the fluid bag 202 to squeeze the fluid bag 202, regardless of the compression state of the sensor door 312 of the pressure sensor assembly 210, are transferred to the force sensor 240 of the pressure sensor assembly 210. These responsive forces are measured by the pressure sensor assembly 210, and are converted or correlated to fluid pressure of the fluid bag 202 by the pressure sensor assembly 210 and/or computer 103 in communication therewith, as described in more detail with reference to FIGS. 4A and 4B.

FIGS. 3C-3D illustrate a cross-sectional view of the pressure sensor assembly 210 in an “uncompressed” and “compressed” position, respectively.

In the uncompressed position of FIG. 3C, the pressure sensor assembly 210 may or may not contact the fluid bag 202, and a lower segment of the sensor door 312 is extended outward, from the opening 326, and towards the fluid bag 202. In particular, in the uncompressed position, outward forces by the springs 302 against the sensor door 312 cause the sensor door 312 to swing about the axis of the pin 316 toward the fluid bag 202, such that the sensor door 312 is not orientated in the same plane as the sensor frame 310, and is not parallel with the plane of the sensor frame 310, as shown by arrow 350. In this position, the springs 302 and the spring bracket 308 are in a semi-compressed state such that the springs 302 are under a predetermined amount of compression pressure, and the spring bracket 308 is not in contact with the sensor door 312 of the pressure sensor assembly 210.

In the compressed position of FIG. 3D, the pressure sensor assembly 210 is pressed against the fluid bag 202 due to lateral translation of the squeeze plate 246 toward the fluid bag 202, and the sensor door 312 is pressed inward (e.g., rotated about the axis of the pin 316 toward the sensor frame 310) by responsive/opposing forces from the fluid bag 202, as shown by arrow 360. For example, the sensor 240 may be considered to be in a compressed state when the pressure sensor assembly 210 is pressed against the fluid bag 202 and the sensor door 312 of the pressure sensor assembly 210 is caused to pivot, or swing, in a direction away from the fluid bag 202 relative to the sensor frame 310 (e.g., as shown by arrow 360). In certain embodiments, in a fully compressed state, the sensor door 312 and sensor frame 310 of the pressure sensor assembly 210 are coplanar.

When the pressure sensor assembly 210 is pressed against the fluid bag 202, the force sensor 240, springs 302, and spring bracket 308 are compressed against the sensor support base 304. In certain embodiments, in the compressed state, the spring bracket 308 may be in contact with the sensor door 312 of the pressure sensor assembly 210.

In certain embodiments, in addition to the pressure sensor assembly 210 monitoring fluid bag pressure, the springs 302 of the pressure sensor assembly 210 are operable to retain energy from mechanical compression for use in compressing the bag in the event of a loss of power to the drive assembly. That is, when (or if) drive assembly 244 loses power or is otherwise unable to laterally translate the squeeze plate 246 against the fluid bag 202 to squeeze the fluid bag 202, the compressed springs 302 will then uncompress and provide squeezing forces against fluid bag 202 to maintain or create a predetermined amount of fluid pressure in fluid bag 202 for a predetermined amount of time (e.g., depending on the arrangement and construction of the springs 302). This arrangement serves as a passive, backup infusion/irrigation mechanism that allows infusion/irrigation to continue for the predetermined amount of time.

FIG. 4A illustrates a detailed cross-sectional view of an exemplary pressure sensor assembly in an uncompressed position. The pressure sensor assembly 210 comprises a sensor support base 304 coupled to the sensor frame 310 of the pressure sensor assembly 210 and in contact with the force sensor 240 positioned between the sensor support base 304 and the sensor door 312 of the pressure sensor assembly 210. The force sensor 240, however, is not in direct contact with the sensor door 312 of the pressure sensor assembly 210, as the spring bracket 308 provides an interface between the force sensor 240 and the sensor door 312 of the pressure sensor assembly 210. The spring bracket 308 and the springs 302 are coupled to the sensor door 312 via spring pins 320.

The spring bracket 308 remains in contact with the force sensor 240 when the pressure sensor assembly 210 is in the uncompressed position (e.g., when the sensor door 312 of the pressure sensor assembly 210 is not in contact with the spring bracket 308). The spring bracket 308 is designed to remain in contact with the force sensor 240, even while the spring force from springs 302 pushes the spring bracket 308 away from the sensor door 312 and against the head 324 of the spring pins 320, so that the force sensor 240 may measure the forces from the fluid bag 202 acting against the sensor door 312 of the pressure sensor assembly 210 even when the pressure sensor assembly 210 is in the uncompressed position.

When a fluid bag 202 is introduced into the fluidics module 101, and the squeeze plate 246 is translated toward the fluid bag 202 to squeeze the fluid bag 202, the fluid bag 202 may provide response forces against the sensor door 312 which are transferred as forces through the springs 302, the spring bracket 308, and to the force sensor 240.

While the described configuration is shown in FIGS. 4A-4B, further configurations are also contemplated, including a spring bracket mounted to the sensor door of the pressure sensor assembly and positioned between the force sensor and the base, or the force sensor positioned such that the portion of the force sensor measuring force is facing the base, for example. The spring bracket, however, may always be in contact with the force sensor in order to monitor the pressure of the fluid bag.

In certain embodiments, the one or more spring pins 320 may be positioned within the springs 302 along the longitudinal axis of the springs 302. The spring pins 320 may be operable to facilitate alignment of the springs 302 with the sensor support base 304 and spring bracket 308, and maintain the longitudinal compression of the springs 302. The springs 302 may be springs as depicted herein (e.g., helical springs as depicted in FIGS. 4A-4B, or flat springs, for example), a biasing member, or a flexing element (e.g., a thin sheet of metal alloy configured to bend in response to mechanical compression). In certain embodiments, the springs 302, spring pins 320, spring bracket 308, and sensor support base 304 may be formed of a metal or metal alloy material such as stainless steel or aluminum, for example.

In the uncompressed position, the sensor door 312 is positioned on a different plane than the sensor frame 310 (e.g., as described in reference to FIG. 3C). The springs 302 are in a semi-compressed state (e.g., not fully compressed) when the pressure sensor assembly 210 is in an uncompressed position. The spring bracket 308 is coupled to the springs 302 disposed around spring pins 320, and the spring bracket 308 is positioned against the head 324 of the spring pins 320 when the pressure sensor assembly 210 is in an uncompressed position. The spring bracket 308, however, is operable to move down the spring pins 320 (e.g., away from the head 324 of the spring pins 320) further compressing the springs 302 when the pressure sensor assembly 210 is compressed, as shown in FIG. 4B.

FIG. 4B illustrates a detailed perspective view of an exemplary pressure sensor assembly in a compressed position. In the compressed position, the sensor door 312 of the pressure sensor assembly 210 is positioned on the same plane as the sensor frame 310. When the pressure sensor assembly 210 is in a compressed state, the springs 302 are further compressed (e.g., thereby storing energy for backup infusion/irrigation). In the compressed position, the spring pins 320 and the spring bracket 308 support the compression of the springs 302, and the spring bracket 308 is further in contact with the back of the sensor door 312 of the pressure sensor assembly 210 to apply pressure to the force sensor 240.

In certain embodiments, as shown in FIGS. 4A-4B, the springs 302 and the spring bracket 308 are coupled to one another at the head 324 of the spring pin 320. When the pressure sensor assembly 210 is translated from an uncompressed position to a compressed position through the pressure from the fluid bag 202 as the squeeze plate 246 is translated toward the fluid bag 202, the spring bracket 308 translates down the spring pin 320 causing further compression of the springs 302. The compression of the springs 302 translates force through the spring bracket 308, and ultimately to the force sensor 240. Accordingly, responsive forces from the fluid bag 202 are applied to the front side of the sensor door 312, which are transferred as force(s) through the springs 302, through the spring bracket 308, and to the force sensor 240.

Additionally, force may be transferred to the force sensor 240 through the compression of the sensor door 312 against the spring bracket 308, which causes the spring bracket 308 to press against the force sensor 240, even in the absence of spring 302 compression. In this sense, forces are also transferred in a parallel force path bypassing the springs 302; from the sensor door 312, to the spring bracket 308, and to the force sensor 240.

In certain other embodiments, the spring bracket 308 may be connected to the pressure sensor assembly 210 similarly to the sensor door 312 of the pressure sensor assembly 210. For example, the spring bracket 308 may be coupled to the pins 316 in the top portion 314 of the pressure sensor assembly 210, similar to the configuration of the sensor door 312 of the pressure sensor assembly 210 as described in relation for FIGS. 3A and 3B. In this embodiment, as the pressure sensor assembly 210 enters a compressed position, the spring bracket 308 would translate about an axis of the pin 316 with the sensor door 312 of the pressure sensor assembly 210 and contact the force sensor 240.

In addition to monitoring fluid bag pressure based on the forces acting on the pressure sensor assembly 210 in response to the squeeze plate 246 being pressed against the fluid bag 202, the pressure sensor assembly 210, and specifically the springs 302, are operable to store energy to continue squeezing the fluid bag 202 upon loss of power to the fluidics system, motor failure, or other loss of compression. For example, once compressed, the springs 302 may be operable to continue providing pressure to the fluid bag 202 to maintain fluid flow to the surgical cassette or the surgical hand piece. The properties of the pressure sensor assembly 210 and squeeze plate 246 may be tailored to provide a desired force pressure for a desired period of time, from one or more different compression positions.

In certain embodiments, the springs 302 may be manufactured to be sized to appropriately retain a specified amount of energy and/or be compressed a specific amount in an uncompressed state and, therefore, deliver a specified amount of force to the fluid bag 202 through the sensor door 312 of the pressure sensor assembly 210. The specified amount of force may directly relate to the distance that the sensor door 312 of the pressure sensor assembly 210 translates towards the fluid bag 202 (e.g., out of plane with the sensor frame 310) upon actuation of the back-up infusion system (e.g., lack of power to the drive assembly or failure of the actuator). The force provided to the fluid bag 202 by the springs 302 in the event of the need of a back-up infusion system (e.g. power loss) may be a specific force to provide the desired back-up infusion parameters according to the type of surgery, or the type of fluid bag, for example.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A back-up infusion pressure sensor module for use in an ophthalmic surgical console, the back-up infusion pressure sensor module comprising:
a squeeze plate configured to engage with a fluid bag;
a pressure sensor assembly, having a sensor door and a sensor frame, wherein the sensor door pivots relative to a pin coupled to a top portion of the pressure sensor assembly, configured to cause the sensor door to be biased towards the fluid bag, the pressure sensor assembly comprising:
a base mounted to a back side of the sensor frame of the pressure sensor assembly;
one or more springs;
a spring bracket coupled to the one or more springs; and
a force sensor in contact with the spring bracket and configured to determine a pressure force from the fluid bag.

2. The back-up infusion pressure sensor module of claim 1, wherein the pressure sensor assembly is configured to have a compressed state and an uncompressed state, wherein:
the pressure sensor assembly is in a compressed state when the sensor door is positioned in the same plane as the sensor frame; and
the pressure sensor assembly is in an uncompressed state when the sensor door is biased towards the fluid bag and the sensor door is not positioned in the same plane as the sensor frame.

3. The back-up infusion pressure sensor module of claim 2, wherein the pressure force from the fluid bag acts on the sensor door of the pressure sensor assembly and the pressure force is transmitted to the force sensor in both the compressed and uncompressed states.

4. The back-up infusion pressure sensor module of claim 2, wherein the spring bracket contacts the back side of the sensor door of the pressure sensor assembly when the pressure sensor assembly is in the compressed state.

5. The back-up infusion pressure sensor module of claim 1, wherein the module further comprises:
an actuator configured to actuate the squeeze plate against the fluid bag, the actuator comprising at least one of: a driver or a leadscrew.

6. The back-up infusion pressure sensor module of claim 1, wherein the pressure sensor assembly is configured to retain energy from mechanical compression of the fluid bag to provide compression force to the fluid bag in the absence of mechanical compression.

7. The back-up infusion pressure sensor module of claim 1, wherein the sensor door of the pressure sensor assembly is coupled to a sensor frame of the pressure sensor assembly with a pin adapted to pivot the sensor door of the pressure sensor assembly about an axis of the pin.

8. A back-up infusion pressure sensor module for use in an ophthalmic surgical console, the back-up infusion pressure sensor module comprising:
a squeeze plate configured to engage with a fluid bag;
a pressure sensor assembly having a first portion and a second portion, wherein the first portion pivots relative to a pin coupled to the second portion and the pressure sensor assembly is configured to cause at least one portion of the pressure sensor assembly to contact the fluid bag, the pressure sensor assembly comprising:
a base mounted to a back side of the pressure sensor assembly;
one or more biasing members;
a bracket coupled to the one or more biasing members; and
a force sensor in contact with the bracket and configured to determine a pressure force from the fluid bag.

9. The back-up infusion pressure sensor module of claim 8, wherein the pressure sensor assembly is configured to have a compressed state and an uncompressed state, wherein:
the pressure sensor assembly is in a compressed state when the first portion of the pressure sensor assembly is positioned in the same plane as the second portion of the pressure sensor assembly; and
the pressure sensor assembly is in an uncompressed state when the first portion of the pressure sensor assembly is biased towards the fluid bag and the first portion is not positioned in the same plane as the second portion.

10. The back-up infusion pressure sensor module of claim 9, wherein the pressure force from the fluid bag acts on the first portion of the pressure sensor assembly and the pressure force is transmitted to the force sensor in both the compressed and uncompressed state.

11. The back-up infusion pressure sensor module of claim 9, wherein the bracket contacts the back side of the first portion of the pressure sensor assembly when the pressure sensor assembly is in the compressed state.

12. The back-up infusion pressure sensor module of claim 8, wherein the module further comprises:
an actuator configured to actuate the squeeze plate against the fluid bag, the actuator comprising at least one of: a driver or a leadscrew.

13. The back-up infusion pressure sensor module of claim 8, wherein the bracket is configured to retain energy from mechanical compression of the fluid bag to provide compression force to the fluid bag in the absence of mechanical compression.

14. The back-up infusion pressure sensor module of claim 8, wherein the first portion of the pressure sensor assembly is coupled to the second portion of the pressure sensor assembly with a pin adapted to pivot the first portion about an axis of the pin.

15. The back-up infusion pressure sensor module of claim 8, wherein the squeeze plate, the first portion of the pressure sensor assembly, and the second portion of the pressure sensor assembly are formed of a stainless steel or aluminum.
